(19) United States
(12) Patent Application Publication
(10) Pub. No.: US 2014/0147493 A1
(43) Pub. Date: May 29, 2014

(54) PCR-BASED GENE DELIVERY CARRIER AND METHOD FOR PREPARING THE SAME

(71) Applicant: KOREA INSTITUTE OF SCIENCE AND TECHNOLOGY, Seoul (KR)

(72) Inventors: Ick Chan KWON, Seoul (KR); Kwang Meyung KIM, Seoul (KR); Sun Hwa KIM, Seoul (KR); In-Cheol SUN, Seoul (KR); Jin Hee NA, Seoul (KR); Sangmin LEE, Seoul (KR); Ju Hee RYU, Seoul (KR)

(73) Assignee: KOREA INSTITUTE OF SCIENCE AND TECHNOLOGY, Seoul (KR)

(21) Appl. No.: 14/071,803

(22) Filed: Nov. 5, 2013

(30) Foreign Application Priority Data
Nov. 29, 2012 (KR) 10-2012-0137013

(51) Int. Cl. A61K 48/00; A61K 9/127

(52) U.S. Cl. CPC A61K 48/0033; A61K 9/127
USPC 424/450; 514/44 R; 424/178.1

(57) ABSTRACT

Disclosed is an efficient carrier for gene delivery to cells based on PCR. The PCR-based gene delivery carrier includes: a shell composed of neutral liposomes; and template DNA and PCR components including polymerase, dNTPs and primers for amplification of the template DNA by PCR, within an inner space defined by the shell. The gene delivery carrier enhances the gene loading efficiency of neutral liposomes without cytotoxicity. Further disclosed is a method for preparing the gene delivery carrier.

### CROSS-REFERENCE TO RELATED APPLICATIONS

This application claims priority under 35 U.S.C. §119 to Korean Patent Application No. 10-2012-0137013 filed on Nov. 29, 2012 in the Korean Intellectual Property Office, the invention of which is incorporated herein by reference in its entirety.

### BACKGROUND OF THE INVENTION

1. Field of the Invention

The present invention relates to an efficient carrier for gene delivery to cells without cytotoxicity, and a method for preparing the same.

2. Description of the Related Art

Over the last few decades, micro- and nano-sized bioreactors consisting of lipid containers have attracted considerable attention as essential tools for understanding the origin of cells in basic life science fields. Since liposome is quite similar in shape and structure to the cellular membrane, liposome-based protocell systems have been extensively studied to perform complex biochemical reaction networks within the closed living compartments.

Recently, several liposome-based models for protocells have been reported to successfully carry out various biochemical reactions, such as photosynthesis, genome replication, and protein synthesis. For instance, Luisi et al. first showed that DNA amplification could take place inside the liposomes via polymerase chain reaction (PCR) technique. They established several critical parameters and conditions to stabilize the liposomes under the high temperature (95° C.) and increase PCR activity inside the liposomes with maintaining the integrity of DNA products.

The compartmentalization of biochemical reactions is also expected to be useful in biotechnological applications including diagnostics and therapeutics. Compared to giant vesicle-based bioreactors (diameter >1 μm), particularly nano-sized protocell systems (hereafter denoted as ‘nano-factories’) may have more opportunities and benefits for the pharmaceutical drug development of bioactive molecules, due to easier access to a single-cell level. Since liposome-based nano-factories can locally amplify various bioactive molecules including DNAs, RNAs and proteins, large amount of therapeutic products can be loaded into the enveloped vesicles using this protocell technique. Certainly, the encapsulated biological materials are protected and stabilized via the guardian lipid barrier in the extracellular environment. Due to this high stability, liposomes have been extensively studied as promising carrier systems for systemic administration of various therapeutic agents.

Up till now, there have been several approaches to the synthesis of oligonucleotides in liposomes, mainly aiming to explore the origin of life. There is no report on the use of the PCR-based nano-factory formulated with neutral lipids as a gene delivery system, especially for in vivo applications. In general, conventional gene carriers including liposomes contain strong cationic charges to efficiently load anionic genetic drugs, such as antisense oligodeoxynucleotide (ODN), plasmid DNA (pDNA) and small interfering RNA (siRNA). The cationic surface charges of gene carriers can also enhance their interaction with anionic cell membranes, leading to increased cellular uptake. Unfortunately, however, this cationic character also leads to destabilization of membranes and causes severe cytotoxicity. Therefore, gene carriers containing cationic charges basically cannot be set free from the toxicity issue, which is the critical hurdle for their clinical applications.

In this connection, Korean Patent Publication No. 2007-36055 discloses a liposome useful for drug delivery. Specifically, the liposome contains a substituted ammonium and/or a polyanion. The patent publication also discloses a liposome composition comprising the liposome and a desired therapeutic or imaging agent. However, the patent publication fails to disclose a liposome for oligonucleic acid synthesis inside the liposome via an amplification technique such as PCR.

### SUMMARY OF THE INVENTION

One object of the present invention is to provide a gene delivery carrier that can markedly enhance the DNA loading efficiency of neutral liposomes without causing cytotoxicity.

Another object of the present invention is to provide a method for preparing the gene delivery carrier.

According to one aspect of the present invention, there is provided a PCR-based gene delivery carrier including: a shell composed of neutral liposomes; and template DNA and PCR components including polymerase, dNTPs and primers for amplification of the template DNA by PCR, within an inner space defined by the shell.

In one embodiment of the present invention, the neutral liposomes may be free from cationic charges.

In a further embodiment of the present invention, the neutral liposomes may include cholesterol, 1,2-dioleoyl-sn-glycero-3-phosphoethanolamine (DOPE), and 1,2-dipalmitoyl-sn-glycero-3-phosphocholine (DPPC).

In another embodiment of the present invention, the PCR-based gene delivery carrier may further include a fluorescent dye to be bound to DNA products obtained after PCR amplification.

In another embodiment of the present invention, the fluorescent dye may be SYBR Green I dye.

In another embodiment of the present invention, the PCR-based gene delivery carrier may be spherical, with a diameter of 50 to 1000 nm.

In another embodiment of the present invention, one or more active targeting moieties selected from the group consisting of antibodies, aptamers, and peptides may be attached to the shell surface.

According to another aspect of the present invention, there is provided a method for preparing a PCR-based gene delivery carrier, the method including: mixing dried lipids in an organic solvent and evaporating the organic solvent to deposit a lipid film; hydrating and dispersing the lipid film in PCR solution containing template DNA and PCR components including polymerase, dNTPs and primers for amplification of the template DNA by PCR, to form multilamellar vesicles; stabilizing morphology of the multilamellar vesicles and repeating freezing and thawing of the multilamellar vesicles to break the multilamellar vesicles into large unilamellar vesicles; passing the large unilamellar vesicles through a filter to homogenize their size; and subjecting the homogenized large unilamellar vesicles to PCR.

In one embodiment of the present invention, the dried lipids may include a mixture of cholesterol, 1,2-dioleoyl-sn-glycero-3-phosphoethanolamine (DOPE), and 1,2-dipalmitoyl-sn-glycero-3-phosphocholine (DPPC).

In a further embodiment of the present invention, the method may further include adding DNase after the homogenization.

In another embodiment of the present invention, the method may further include, after the homogenization, adding a fluorescent dye to be bound to DNA products amplified by the PCR.

In another embodiment of the present invention, 5 to 30 cycles of freezing and thawing may be repeated.

In another embodiment of the present invention, the filter may include pores having a diameter of 0.1 to 0.8 μm.

In another embodiment of the present invention, the PCR may include i) incubating at 90 to 100° C. for 0.5 to 5 minutes, and ii) incubating at 90 to 100° C. for 10 seconds to 3 minutes and incubating at 50 to 80° C. for 10 seconds to 5 minutes, which may be repeated 10 to 40 times.

In an alternative embodiment of the present invention, the PCR may include i) incubating at 90 to 100° C. for 0.5 to 5 minutes, and ii) incubating at 90 to 100° C. for 10 seconds to 3 minutes, incubating at 50 to 70° C. for 10 seconds to 5 minutes, and incubating at 60 to 80° C. for 10 seconds to 5 minutes, which may be repeated 10 to 40 times.

The gene delivery carrier of the present invention enhances the gene loading efficiency of neutral liposomes without cytotoxicity. In addition, the method of the present invention is suitable for the preparation of the gene delivery carrier.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic illustration of a gene delivery carrier according to the present invention;

FIG. 2 shows the chemical formulae of cholesterol, 1,2-dioleoyl-sn-glycero-3-phosphoethanolamine (DOPE), and 1,2-dipalmitoyl-sn-glycero-3-phosphocholine (DPPC) as exemplary components constituting neutral liposomes of a gene delivery carrier according to the present invention;

FIG. 3a shows particle size distributions measured by DLS before and after PCR amplification in a gene delivery carrier of the present invention, and the insets show CryoTEM images;

FIG. 3b shows images of a gene delivery carrier of the present invention (1) before and (2) after 20 PCR thermal cycles;

FIG. 3c shows an agarose gel image of amplified linear DNA oligomers in a gene delivery carrier of the present invention (Lane 1, 2 and 3 represent PCR mixtured-liposomes and PCR-amplified-liposomes with and without pDNA template, respectively);

FIG. 3d shows sequences of template plasmid DNA and primers used to amplify DNA in a gene delivery carrier of the present invention;

FIG. 3e shows images of transfected cells at 48 hr post-transfection of amplified linear DNA oligomers with Lipo2K into CHO-K1 cells;

FIG. 4a shows changes in cell viability depending on the concentration of a gene delivery carrier of the present invention;

FIG. 4b shows the gene transfection efficiency of CHO-K1 cells after transfection with various DNA formulations;

FIG. 4c shows representative fluorescence microscopy images for eGFP gene transfected cells with various DNA formulations; and

FIGS. 5a to 5e shows comparative analysis data between two different DNA loading techniques: FIG. 5a is an agarose gel image; FIG. 5b shows gene transfection efficiency in CHO-K1 cells (1 and 2: encapsulation of linear DNA oligomers in neutral liposomes after general PCR amplification in aqueous phase, and 3 and 4: DNA amplification inside neutral liposomes via a gene delivery carrier of the present invention. The DNA-loaded liposomes were further treated with DNase (2 and 4) or not (1 and 3). M: DNA size marker); and FIGS. 5c to 5e are images showing in vivo gene expression of a gene delivery carrier of the present invention containing DsRed2 as a target gene.

### DETAILED DESCRIPTION OF THE INVENTION

The present invention provides a PCR-based gene delivery carrier using a liposome-based protocell system, particularly a gene delivery carrier capable of efficient gene loading without causing any problems, such as destabilization of membranes or cytotoxicity.

Specifically, the PCR-based gene delivery carrier of the present invention includes: a shell composed of neutral liposomes; and template DNA and PCR components including polymerase, dNTPs and primers for amplification of the template DNA by PCR, within an inner space defined by the shell.

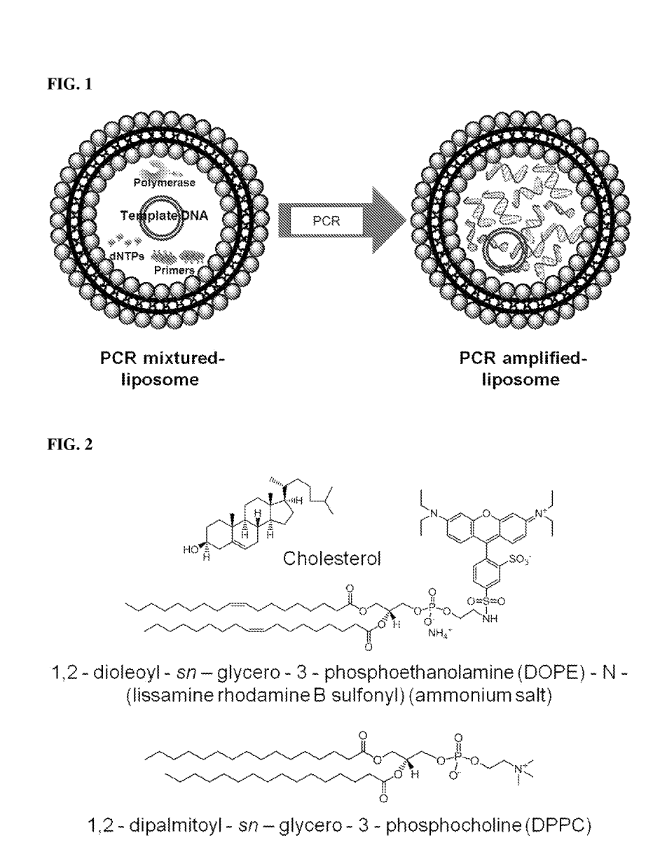

FIG. 1 is a schematic illustration of the gene delivery carrier according to the present invention. Referring to FIG. 1, the gene delivery carrier of the present invention has a structure in which the template DNA as a target for PCR amplification, the polymerase for catalyzing the PCR amplification, the dNTPs as raw bases for amplification by the polymerase, and the primers for initialization of the polymerization are contained within an inner space of the shell composed of neutral liposomes.

The neutral liposomes are preferably free from cationic charges. Cationic charges of the neutral liposomes lead to destabilization of membranes and cause severe cytotoxicity, as described above.

Specifically, the neutral liposomes may include cholesterol, 1,2-dioleoyl-sn-glycero-3-phosphoethanolamine (DOPE), and 1,2-dipalmitoyl-sn-glycero-3-phosphocholine (DPPC).

The PCR-based gene delivery carrier of the present invention may further include a fluorescent dye to be bound to DNA products obtained after PCR amplification. In this case, the gene delivery carrier can be advantageously monitored in vitro and/or in vivo via the fluorescence from the DNA-fluorescent dye complexes. A specific non-limiting example of the fluorescent dye may be SYBR Green I dye.

As can be seen from the Examples Section that follows, the PCR-based gene delivery carrier is spherical, with a diameter of 50 to 1000 nm. One or more active targeting moieties such as antibodies, aptamers, and peptides may be attached to the surface of the spherical shell to modify the surface of the neutral liposomes. This surface modification can further improve the transfection efficiency of the PCR-based gene delivery carrier both in vitro and in vivo.

The present invention also provides a method for preparing the PCR-based gene delivery carrier. The method includes: mixing dried lipids in an organic solvent and evaporating the organic solvent to deposit a lipid film; hydrating and dispersing the lipid film in PCR solution containing template DNA and PCR components including polymerase, dNTPs and primers for amplification of the template DNA by PCR, to form multilamellar vesicles; stabilizing morphology of the multilamellar vesicles and repeating freezing and thawing of the multilamellar vesicles to break the multilamellar vesicles into large unilamellar vesicles; passing the large unilamellar vesicles through a filter to homogenize their size; and subjecting the homogenized large unilamellar vesicles to PCR.

As described above, the dried lipids are neutral lipids free from cationic charges. For example, the dried lipids may be composed of a mixture of cholesterol, 1,2-dioleoyl-sn-glycero-3-phosphoethanolamine (DOPE), and 1,2-dipalmitoyl-sn-glycero-3-phosphocholine (DPPC).

After the homogenization, DNase may be further added to exclude the possibility of undesirable DNA oligomers amplified outside of the liposomes. Specifically, as the DNase, there may be mentioned, for example, DNase I.

As described previously, a fluorescent dye may be further added after the homogenization. The fluorescent dye is bound to DNA products amplified by the PCR and enables in vitro and/or in vivo monitoring of the gene delivery carrier of the present invention.

The freezing and thawing is conducted to break the multilamellar vesicles (MLVs) formed by hydrating and dispersing the lipid film in PCR solution. The freezing may be conducted using liquid nitrogen and the thawing may be conducted by heating above the transition temperature of the lipids. 5 to 30 cycles of freezing and thawing may be applied. At less than 5 cycles of freezing and thawing, it is impossible to break the multilamellar vesicles to a sufficient extent. Meanwhile, cycles of freezing and thawing exceeding 30 cycles are meaningless because most of the multilamellar vesicles are already broken.

Subsequently, the large unilamellar vesicles are filtered using a filter to achieve size homogenization of the large unilamellar vesicles. This filtration is needed because the large unilamellar vesicles exist in the form of a mixture of vesicles having various sizes. The filter may include pores having a diameter of 0.1 to 0.8 μm. If the pore diameter is less than 0.1 μm, most of the vesicles cannot pass through the filter, resulting in an excessively low yield of the large unilamellar vesicles, and very small vesicles only are obtained. Meanwhile, if the pore diameter exceeds 0.8 μm, vesicles of very various sizes can pass through the filter, failing to achieve desired size homogenization.

Finally, PCR is conducted inside the large unilamellar vesicles in which the PCR reagents are loaded into the neutral liposomes. As a result of PCR, DNA amplified inside the liposomes. At this time, the PCR may be conducted, for example, by incubating the large unilamellar vesicles at 90 to 100° C. for 0.5 to 5 minutes, followed by 10 to 40 cycles of incubations at 90 to 100° C. for 10 seconds to 3 minutes and at 50 to 80° C. for 10 seconds to 5 minutes. Alternatively, the PCR may be conducted, for example, by incubating the large unilamellar vesicles at 90 to 100° C. for 0.5 to 5 minutes, followed by 10 to 40 cycles of incubations at 90 to 100° C. for 10 seconds to 3 minutes, at 50 to 70° C. for 10 seconds to 5 minutes, and at 60 to 80° C. for 10 seconds to minutes.

The present invention will be explained in more detail with reference to the following examples. These examples are not intended to limit the scope of the invention and are provided to assist in understanding the invention.

Materials

1,2-dipalmitoyl-sn-glycero-3-phosphocholine (DPPC), 1,2-dioleoyl-sn-glycero-3-phosphoethanolamine-N-(lissaminerhodamine B sulfonyl) ammonium salt (DOPE-Rhod), Cholesterol (CHOL) were purchased from Avanti Polar Lipids (Alabaster, Ala., USA). Template plasmid DNA (pCMV-EGFP, pCMV-DsRed2, pGL3-Luciferase) was obtained from Clontech Laboratories (Palo Alto, Calif., USA) and Promega (Madison, Wis., USA). DNA primers were purchased from Bioneer (Daejeon, Korea). HiPi™Thermostable DNA polymerase and DNase I were bought from ElpisBio (Daejeon, Korea) and Takara (Kyoto, Japan), respectively. SYBR Green I dye and Lipofectamine™2000 were purchased from Invitrogen (Carlsbad, Calif., USA). Gel Extraction kit and PCR purification kit were purchased from Qiagen (Chatsworth, Calif., USA). Branched polyethylenimine (BPEI) with an average molecular weight of 25 kDa was obtained from Sigma-Aldrich (St. Louis, Mo., USA). All other chemicals were of analytical grade and used without further purification.

PCR Solution

The PCR solution (1 ml) contained deionized water (720 μl), PCR buffer solution (10× HiPi™ buffer, 100 μl), dNTP mix (2 mM each dNTP in TE buffer, 100 μl), 21-mer primers (5′-Cy5 fluorescent dye modified, 10 μM, 20 μl, each), plasmid DNA template (10 nM, 20 μl), HiPi™Thermostable DNA polymerase (1Uμl⁻¹, 20 μl), and 0.5×SYBR Green I dye (commercial product diluted 10,000× in TE buffer, 10 μl).

Preparation of Liposome (Nano-Factory System)

Liposomes were prepared using the film hydration method. Dried lipids were mixed in chloroform in glass vial with the following composition (DPPC:CHOL:DOPE-Rhod=13:6:1 in molar ratio, total amount of lipid was 2 μmole/sample). The organic solvent was evaporated by rotary evaporator resulting in the deposition of a thin lipid film on the glass vial wall. The lipid film was freeze-dried overnight to remove traces of remaining organic solvent and then hydrated and dispersed in PCR solution (1 ml) by vortex mixing. Following gentle pipetting manipulation formed multilamellar vesicles (MLVs) encapsulating the PCR reagents, the MLVs dispersion was left for 30 min at room temperature in order to stabilize morphology of the MLVs. To break the MLVs into large unilamellar vesicles (LUVs), ten cycles of freezing (liquid nitrogen) and thawing (above the transition temperature of the lipids) were applied. Size of liposome was homogenized by extrusion by passing the sample 10 fold through a 200 nm pore cellulose acetate filter. DNase I of 0.002 U/μl in 1×PCR buffer containing 0.5×SYBR Green I dye was added to the liposome dispersion in order to digest the DNA template and primers in exterior the liposome (for 5 min, at room temperature).

Protocol of Thermal Cycling

The nano-factory system was treated with a thermal cycler (Veriti® thermal cycler, Applied Biosystems, Foster City, Calif., USA) under following thermal conditions: 94° C. for 2 min, [94° C. for 15 sec and 68° C. for 1.5 min]×20 cycles.

Characterization of Liposome (Nano-Factory System)

The morphology of liposomes was observed by cryogenic transmission electron microscope (cryo-TEM). Each sample was prepared as a thin aqueous film supported on a holey-carbon grid. Cryo-TEM images were obtained at a temperature of approximately −170° C. with a 200 kV Tecnai F20 (FEI, Netherlands). The average diameter, size distribution and the surface charge of liposomes were determined using a Zetasizer Nano ZS (Malvern Instruments, Worcestershire, UK).

Detection of Amplified Linear DNA Oligomers

The PCR-mixtured liposome dispersion and PCR-amplified liposome dispersion were transferred into 96-well plates and the amplification of DNA by PCR was analyzed by fluorescence with a 12-bit CCD camera (Kodak Image Station 4000 MM, New Haven, Conn., USA) equipped with a special C-mount lens and filter set for FITC and TRITC.

Amplified linear DNA oligomers of the liposome dispersion were retrieved by the following procedure. A mixture of 150 μl TE-saturated phenol-chloroform-iso-amylalcohol (25/24/1, v/v/v) was added to the PCR-amplified liposome dispersion after DNase I treatment (150 μl), and the lipids and enzymes were removed from the buffered solution containing the amplified linear DNA oligomers. Amplified linear DNA oligomers in the resulting aqueous solution were purified using the PCR purification kit according to the manufacturer's protocol. The purified DNA oligomers were analyzed by agarose gel (1.0%) electrophoresis.

Cell Culture

The Chinese Hamster Ovary cells (CHO-K1, purchased from ATCC, Manassas, Va., USA) were maintained in DMEM (Welgene, Daegu, Korea), supplemented with 10% fetal bovine serum (FBS; Welgene, Daegu, Korea), 100 U/ml penicillin and 100 μg/ml streptomycin (Welgene, Daegu, Korea) at 37° C. in a humidified 5% CO₂ atmosphere.

Gene Expression Test of Amplified Linear DNA Oligomers

The amplified linear DNA oligomers were purified with the gel extraction kit (Qiagen, Chatsworth, Calif., USA) and the DNA complex with Lipofectamine™2000 (Lipo2k) was prepared according to manufacturer's protocol. CHO-K1 cells were seeded onto 35 mm glass-bottom dish at a density of 5×10⁴ cells in 2 ml of serum-free medium and grown to reach 60% confluence. The culture medium was replaced with 2 ml of the transfection medium containing DNA complex with Lipo2k, followed by 6 hour-incubation at 37° C. The transfection medium was then replaced with the fresh complete DMEM medium (10% FBS), and the cells were allowed to grow for 48 hours. After incubation, the cells were washed twice with PBS (pH 7.4) containing Ca²⁺ and Mg²⁺, fixed with formaldehyde-glutaraldehyde combined fixative for 15 min at room temperature, and then stained with DAPI (Invitrogen, Carlsbad, Calif., USA) to label nuclei. All cellular images were obtained using IX81-ZDC focus drift compensating microscope (Olympus, Tokyo, Japan).

Cellular Uptake of Nano-Factory System

CHO-K1 cells were seeded onto 35 mm glass-bottom dish and allowed to grow until a confluence 60-80%. Then, the cells were washed twice with PBS (pH 7.4) to remove the remnant growth medium. The cells were incubated with nano-factory system at concentration of 200 μg/ml for up to 4 hours at 37° C. in 2 ml serum-free medium. Then, they were washed twice with PBS containing Ca²⁺ and Mg²⁺, fixed with formaldehyde-glutaraldehyde combined fixative for 15 min at room temperature, and then stained with DAPI (Invitrogen, Carlsbad, Calif., USA) to label nuclei.

Cytotoxicity Assay

The cytotoxicity of the nano-factory system according to the present invention was evaluated with the MTT assay. CHO-K1 cells were seeded in 96-well plates at an initial density of 5×10³ cells per well in 200 μl of the complete medium. After 24 hours, the medium was replaced with 200 μl of fresh complete medium, to which BPEI (25 kDa), Lipofectamine™2000 (Lipo2k) or the nano-factory system according to the present invention was added to achieve carrier concentration from 1 μg/ml to 200 μg/ml. After 24 hours of incubation, 25 μl of the (3-(4,5-dimethylthiazol-2-yl)-2,5-diphenyl-tetrazolium bromide) (MTT) reagent (5 mg/ml in media) was added to each well and in the absence of light, the cells were incubated for 2 hours at 37° C. And then 200 μl of DMSO was added to each well. Absorbance at 570 nm was measured with a microplate reader (VERSAmax™, Molecular Devices Corp., Sunnyvale, Calif., USA).

In Vitro Gene Transfection

For comparison, a DNA complex with Lipo2k was prepared according to manufacturer's protocol and BPEI (25 kDa) was prepared at the N/P ratio of 20/1. CHO-K1 cells were seeded onto 35 mm glass-bottom dish at a density of 5×10⁴ cells in 2 ml of serum-free medium and grown to reach 60% confluence. The culture medium was replaced with 2 ml of the transfection medium containing BPEI, Lipo2k, and the nano-factory system of the present invention (equivalent to 100 ng template plasmid DNA), followed by 6 hour-incubation at 37° C. The transfection medium was then replaced with the fresh complete DMEM medium (10% FBS), and the cells were allowed to grow for 48 hours. After incubation, the cells were washed twice with PBS (pH 7.4) containing Ca²⁺ and Mg²⁺, fixed with formaldehyde-glutaraldehyde combined fixative for 15 min at room temperature.

Quantitative assay was done as follows: The cells were seeded onto 6-well plate at a density of 5×10⁵ cells in 2 mL of media and grown to reach 60-80% confluence. The culture medium was replaced with 2 ml of the transfection medium containing BPEI, Lipo2k, and the nano-factory system of the present invention (equivalent to 100 ng template plasmid DNA), followed by 6 hour-incubation at 37° C. The transfection medium was then replaced with the fresh complete DMEM medium (10% FBS), and the cells were allowed to grow for 48 hours. They were washed with PBS (pH 7.4), lysed with the cell lysis buffer (Sigma-Aldrich, St. Louis, Mo., USA). Total soluble protein concentration was determined by bicinchoninic acid (BCA) protein assay (Pierce, Ill., USA). Fluorescence intensity of the samples was measured by use of a fluorescence spectrophotometer (Hitachi F-7000, Tokyo, Japan). The fluorescence intensity was normalized by dividing with the amount of proteins determined in BCA assay. To evaluate enhanced DNA loading capacity of neutral liposome via PCR-based nano-factory, same method was used.

Results and Discussion

The present invention provides nano-sized liposomes encapsulating pDNA template and PCR components including polymerase, primers and dNTPs. The nano-sized liposomes were made using the film hydration and the freeze-thaw method with subsequent extrusion, immediately followed by conventional 20 PCR cycles (FIG. 1).

In order to visualize exogenous gene expression in vitro and in vivo respectively, enhanced green fluorescent protein (eGFP), red fluorescent protein (DsRed2) and luminescence emitting enzyme protein (luciferase) were used as reporter target genes. Each target pDNA template was loaded into the neutral liposomes composed of cholesterol, 1,2-dioleoyl-sn-glycero-3-phosphoethanolamine (DOPE) and 1,2-dipalmitoyl-sn-glycero-3-phosphocholine (DPPC) (FIG. 2).

To prepare PCR-based nano-factories, first the lipid film comprised of a mixture of aforementioned amphiphiles was hydrated and dispersed with PCR solution containing pDNA template, polymerase, primers and dNTPs. In particular, SYBR Green I dye was included in PCR solution for visualizing produced target DNA oligomers after PCR amplification via the fluorescence from the SYBR Green I dye/DNA complexes.

After the formation of large unilamellar vesicles via 10 freeze-thaw cycles, the heterogeneous populations of liposomes were passed through cellulose acetate membrane filters with a diameter of 200 nm to yield the desired homogeneous liposome populations. To exclude the possibility of undesirable DNA oligomers amplified outside of the liposomes, the pDNA template remaining in the exterior aqueous phase was removed via DNase I digestion.

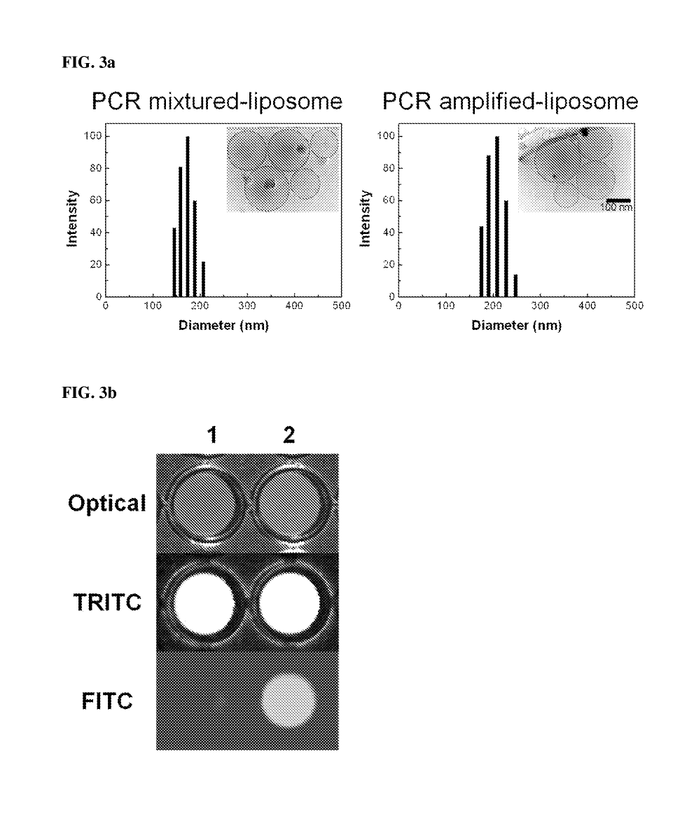

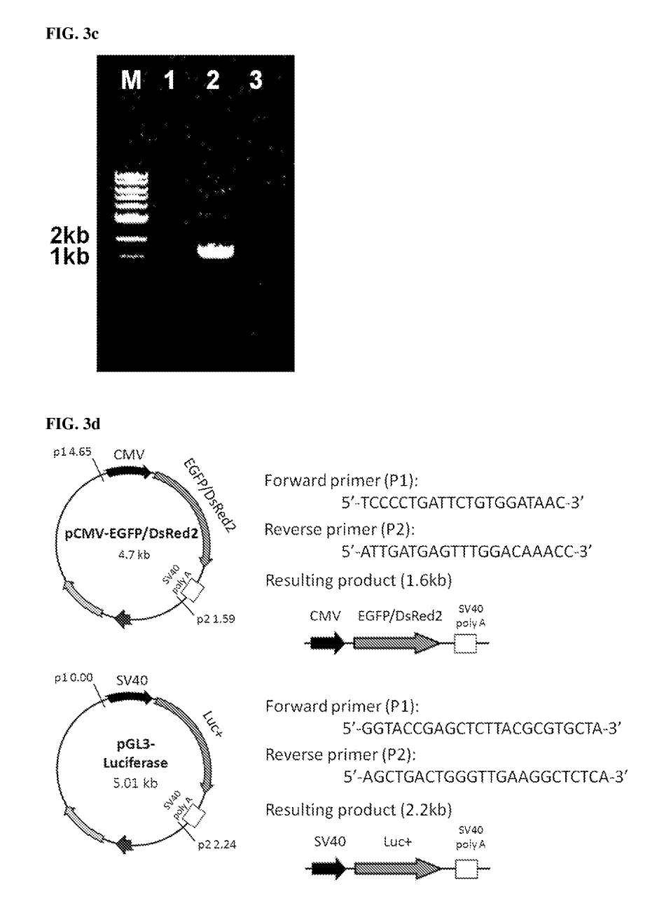

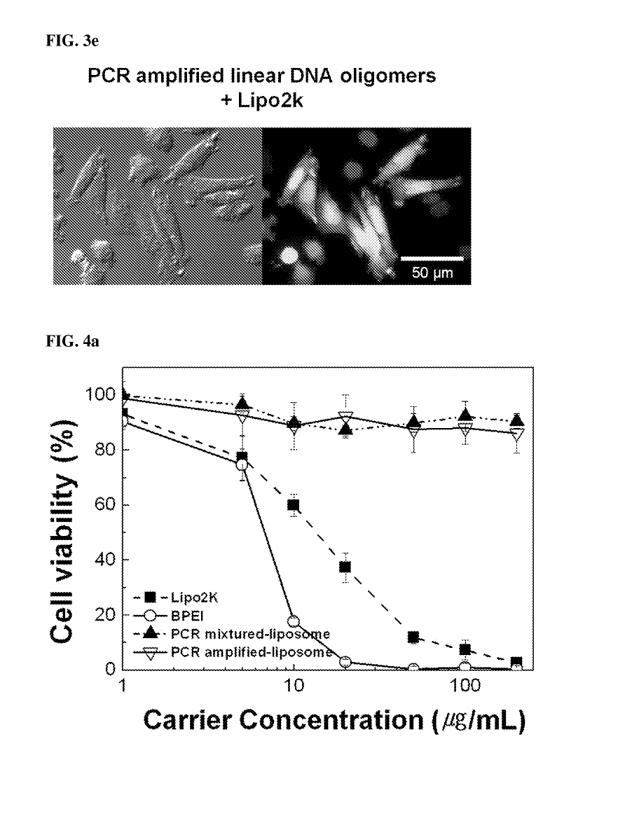

After DNA amplification, the PCR-based nano-factory of the present invention showed spherical shape of liposomes with 150-250 nm in particle size (FIG. 3a). There was no significant difference in the size and shape of liposomes before and after the PCR amplification. These results strongly support that the PCR-based nano-factory of the present invention could remain stable during the high-temperature PCR phases.

Indeed, the empty liposomes exhibited a neutral surface charge. After encapsulating pDNA template and conducting the PCR reaction, however, the resulting liposomes possessed slightly negative surface charge (ζ-potential=−3-8 and −3-10 mV, respectively), which might be attributed to the existence of anionic pDNA cargoes smeared on the liposome surface.

This result supports that the PCR-based nano-factory of the present invention using a neutral liposome-DNA formulation is totally free from cationic charges without electrostatic interactions between opposite charges. However, the neutral liposomes naturally decrease DNA entrapment efficiency due to the absence of charge-charge interaction. Despite the non-toxic nature of neutral lipids, thus the low DNA loading efficiency has been a potential drag on neutral liposome-mediated gene delivery.

Thus, it was hypothesized that increasing DNA concentration within the liposomes via PCR amplification may overcome the low DNA loading capacity of neutral liposomes. The amplification of DNA oligomers by PCR method was examined using the fluorescent signals for the SYBR Green I dye/DNA complexes. The PCR-based nano-factory of the present invention exhibited intense green fluorescence after conducting the PCR (FIG. 3b), clearly indicating the DNA fragments were successfully amplified in the artificial cell model of the present invention.

In addition, the exact length of DNA fragments produced in the PCR-based nano-factory of the present invention was further investigated by agarose gel electrophoresis (FIG. 3c). Based on the pDNA template containing CMV promoter and eGFP/DsRed2 or SV40 promoter and luciferase gene sequences, the amplified DNA oligomers are expected to be 1.6 kb in length under general PCR amplification with rationally designed primers (FIG. 3d). Indeed, the resulting 1.6 kb linear DNA products were obtained.

In addition, the DNA products amplified inside the liposomes were still remained even after DNase digestion, while the DNA oligomers produced via general PCR amplification were completely degraded during DNase treatment. This result demonstrates that the PCR-based nano-factory technique of the present invention allows the amplified DNA oligomers to keep serum stability in blood conditions.

To test the gene expression ability of the amplified linear DNA oligomers, they were transfected with a traditional gene carrier Lipofectamine™2000 (Lip2K) into CHO-K1 cells. At 48 hr post-transfection, the intense green fluorescence signals were observed in the transfected cells, certainly showing the gene expression induced by the amplified DNA oligomers (FIG. 3e).

In order to evaluate the applicability of the PCR-based nano-factory according to the present invention to gene therapy, its cellular toxicity and in vitro gene delivery efficiency were assessed before and after PCR amplification in comparison with those of conventional cationic gene carriers, such as BPEI and Lipo2K.

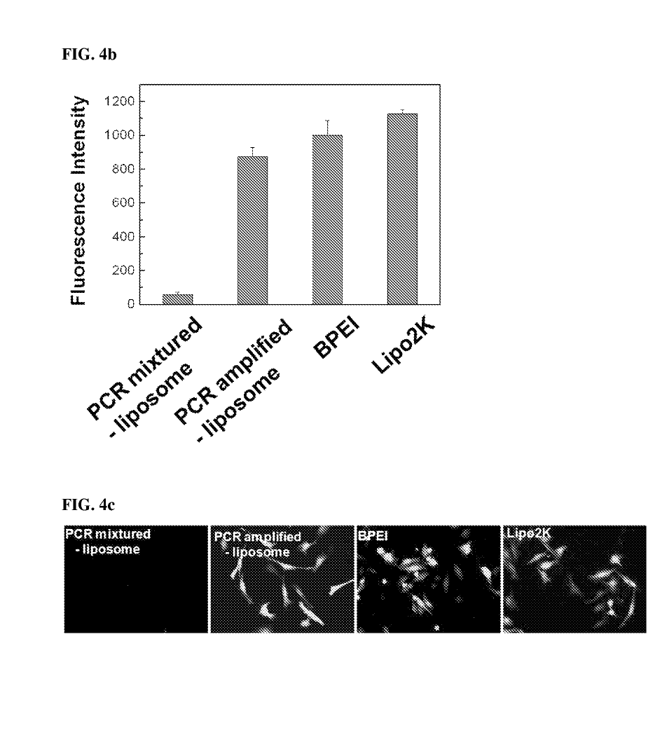

As expected, conventional transfection reagents BPEI and Lipo2K exhibited severe cytotoxicity against CHO-K1 cells (IC₅₀ values: 5-10 and 10-15 μg/mL, respectively, see FIG. 4a), mainly due to their strong cationic charges. On the other hand, neutral lipid-based liposomes with and without PCR amplification showed negligible cytotoxicity even at extremely high carrier concentrations (≧200 μg/mL).

This result demonstrates the superior safety of the PCR-based nano-factory system according to the present invention, which might be mainly attributed to its neutral nature.

In vitro gene transfection efficiency of the PCR-based nano-factory according to the present invention was also evaluated in CHO-K1 cells using pDNA template containing eGFP gene (FIGS. 4b and 4c). Premixture-liposome formulation showed a very weak green fluorescent signal, which resulted from the liposome-entrapped pDNA template though the amount of target genes was obviously insufficient for efficient transfection. After PCR amplification, however, the cells transfected with PCR-based nano-factory of the present invention exhibited vivid green fluorescence.

Interestingly, the nano-factory formulation of the present invention achieved a high level of transfection efficiency similar to those of traditional transfection reagents such as PBEI and LIpo2K, indicating that the enhancement of transfection efficiency of neutral liposomes generated by the amplified linear DNA oligomers, not by pDNA template. Specifically, the relative fluorescence intensity values of PCR mixture and PCR amplified-liposomes, BPEI and Lipo2K were 40-70, 820-930, 920-1100, and 1100-1150, respectively. Although cationic lipid reagents seem to have a potential as gene carriers with high transfection efficiency, the toxicity issue inevitably limits their use in clinical applications, as described above.

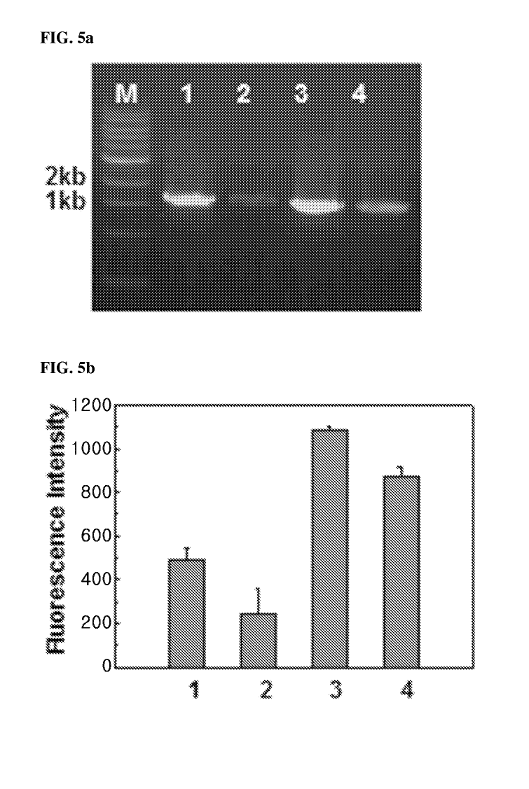

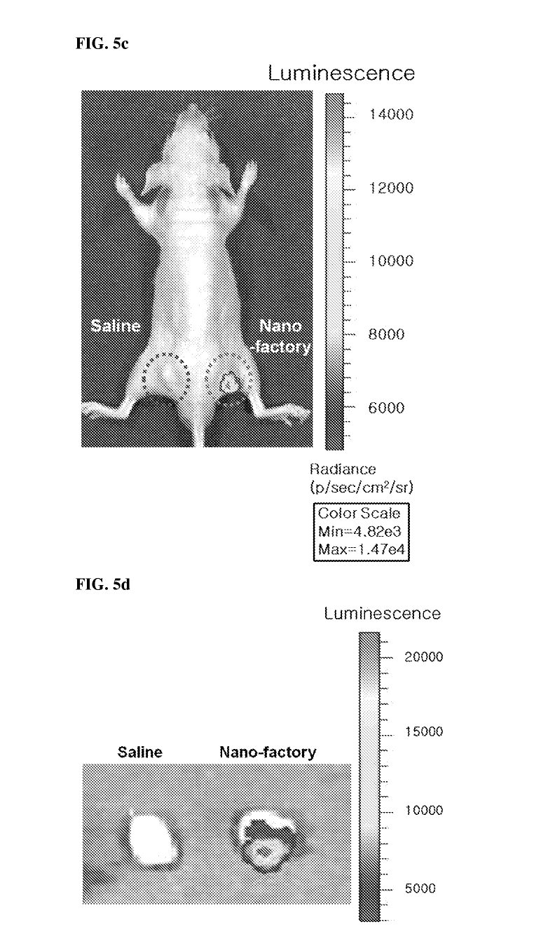

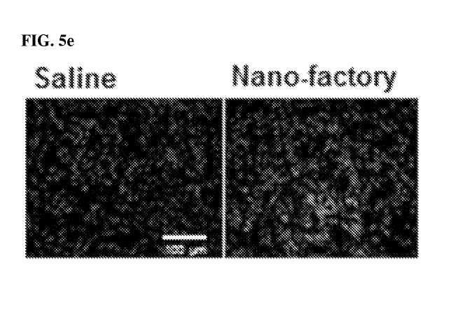

To evaluate enhanced DNA loading capacity of neutral liposome via PCR-based nano-factory, the effect of two different DNA encapsulation methods on the transfection efficiency was directly compared, when the target DNA molecules were loaded into the neutral liposomes before and after PCR amplification (FIGS. 5a and 5b).

As expected, the PCR-based nano-factory formulation showed much higher DNA-loading efficiency than the post-encapsulation of DNA products obtained after PCR amplification in aqueous phase. Due to the absence of electrostatic interactions of oppositely charged molecules, the amplified DNA products were not efficiently encapsulated in neutral liposomes and were present outside the liposomes, which resulted in a rapid enzymatic degradation of DNA oligomers. However, the DNA molecules amplified inside the liposomes could remain intact even after DNase attack. This result clearly suggests that the PCR-based nano-factory method of the present invention is more effective for encapsulating negatively charged DNA drugs into neutral liposomes without toxic cationic lipids, compared to the traditional liposomal transfection methods. As a result, there were big differences in gene transfection efficiency depending on the target DNA encapsulation methods. The PCR-based nano-factory system of the present invention (840-920) exhibited more than 4-fold increase in relative fluorescence intensity compared to the post-encapsulation of amplified DNA oligomers in neutral liposomes (130-360), which was shown to tightly correlate with the DNA loading capacity. These results demonstrate that PCR-based nano-factory technique of the present invention could be an alternative approach for efficient encapsulation of negatively charged DNA drugs in neutral liposomes.

Finally, in vivo gene expression with the PCR-based nano-factory system of the present invention was evaluated. For in vivo studies, xenograft mice models bearing tumors in both side of flank were prepared by subcutaneous injection of A549-Luc cells. Then, the PCR-based nano-factory was administered to the right tumor by intratumoral injection once daily for 2 days, while saline was injected to the left one as a control. In vivo gene expression with the nano-factory system in the tumors was observed by luminescence (FIGS. 5c and 5d). The fluorescence microscope images of tumor tissues showed intense red fluorescence in case of the PCR-based nano-factory (FIG. 5e).

In conclusion, the PCR-based nano-factory was developed as a safe gene delivery system in the present invention. A few template plasmid DNA can be amplified by PCR inside liposomes and the amount of loaded genes highly increased. The shell liposome was composed of neutral lipids free from cationic charges. Consequently, the system of the present invention is non-toxic in different with other traditional cationic gene carriers. Intense GFP expression in CHO cells showed the amplified genes were successfully transfected to cells.

Therefore, the PCR-based nano-factory system of the present invention can overcome the toxicity problem which is the critical hurdle of current gene delivery for clinical application. Furthermore, surface modification of neutral liposomes via adding various active targeting moieties such as antibodies, aptamers and peptides can further improve the transfection efficiency of the PCR-based nano-factory system according to the present invention both in vitro and in vivo.

What is claimed is:

1. A PCR-based gene delivery carrier comprising:
a shell composed of neutral liposomes; and
template DNA and PCR components comprising polymerase, dNTPs and primers for amplification of the template DNA by PCR, within an inner space defined by the shell.

2. The PCR-based gene delivery carrier according to claim 1, wherein the neutral liposomes are free from cationic charges.

3. The PCR-based gene delivery carrier according to claim 2, wherein the neutral liposomes comprise cholesterol, 1,2-dioleoyl-sn-glycero-3-phosphoethanolamine (DOPE), and 1,2-dipalmitoyl-sn-glycero-3-phosphocholine (DPPC).

4. The PCR-based gene delivery carrier according to claim 1, further comprising a fluorescent dye to be bound to DNA products obtained after PCR amplification.

5. The PCR-based gene delivery carrier according to claim 4, wherein the fluorescent dye is SYBR Green I dye.

6. The PCR-based gene delivery carrier according to claim 1, wherein the PCR-based gene delivery carrier is spherical, with a diameter of 50 to 1000 nm.

7. The PCR-based gene delivery carrier according to claim 1, wherein one or more active targeting moieties selected from the group consisting of antibodies, aptamers, and peptides are attached to the shell surface.

8. A method for preparing a PCR-based gene delivery carrier, the method comprising:
mixing dried lipids in an organic solvent and evaporating the organic solvent to deposit a lipid film;
hydrating and dispersing the lipid film in PCR solution containing template DNA and PCR components comprising polymerase, dNTPs and primers for amplification of the template DNA by PCR, to form multilamellar vesicles;
stabilizing morphology of the multilamellar vesicles and repeating freezing and thawing of the multilamellar vesicles to break the multilamellar vesicles into large unilamellar vesicles;
passing the large unilamellar vesicles through a filter to homogenize their size; and
subjecting the homogenized large unilamellar vesicles to PCR.

9. The method according to claim 8, wherein the dried lipids comprise a mixture of cholesterol, 1,2-dioleoyl-sn-glycero-3-phosphoethanolamine (DOPE), and 1,2-dipalmitoyl-sn-glycero-3-phosphocholine (DPPC).

10. The method according to claim 8, further comprising adding DNase after the homogenization.

11. The method according to claim 8, further comprising, after the homogenization, adding a fluorescent dye to be bound to DNA products amplified by the PCR.

12. The method according to claim 8, wherein 5 to 30 cycles of freezing and thawing are repeated.

13. The method according to claim 8, wherein the filter comprises pores having a diameter of 0.1 to 0.8 μm.

14. The method according to claim 8, wherein the PCR comprises
i) incubating at 90 to 100° C. for 0.5 to 5 minutes, and
ii) incubating at 90 to 100° C. for 10 seconds to 3 minutes and incubating at 50 to 80° C. for 10 seconds to 5 minutes, which are repeated 10 to 40 times.

15. The method according to claim 8, wherein the PCR comprises
i) incubating at 90 to 100° C. for 0.5 to 5 minutes, and
ii) incubating at 90 to 100° C. for 10 seconds to 3 minutes, incubating at 50 to 70° C. for 10 seconds to 5 minutes, and incubating at 60 to 80° C. for 10 seconds to 5 minutes, which are repeated 10 to 40 times.
